(19) United States
(12) Patent Application Publication
(10) Pub. No.: US 2025/0041095 A1
(43) Pub. Date: Feb. 6, 2025

(54) APPARATUS AND METHOD FOR SEALING THE ORAL CAVITY TO PROMOTE NASAL BREATHING

(71) Applicant: SomnoSeal, Portland, OR (US)

(72) Inventor: Kimberly Hutchison, Portland, OR (US)

(73) Assignee: SomnoSeal, Portland, OR (US)

(21) Appl. No.: 18/680,683

(22) Filed: May 31, 2024

Related U.S. Application Data
(60) Provisional application No. 63/530,439, filed on Aug. 2, 2023.

(51) Int. Cl. A61F 5/08; A61M 16/06

(52) U.S. Cl. CPC A61F 5/08; A61M 16/0683; A61M 2210/0618

(57) ABSTRACT

An apparatus for sealing the oral cavity of a subject to promote nasal breathing, comprising a body having, a front surface, a rear surface, wherein the rear surface is positioned opposite the front surface, a perimeter, wherein the perimeter defines the boundary between the front surface and the rear surface, a first side, a second side, wherein the first side and the second side are located on opposite edges of the body, a lateral axis running from the first side to the second side, a first end, a second end, wherein the first end and the second end are located on opposite edges of the body, and a longitudinal axis running from the first end to the second end, wherein the body is configured to fit into a vestibular oral cavity of a subject, and a rim, wherein the rim is coupled to the perimeter of the body.

### CROSS-REFERENCE TO RELATED APPLICATIONS

This application claims the benefit of priority of U.S. Provisional Patent Application Ser. No. 63/530,439, filed on Aug. 2, 2023, and titled “ORAL APPLIANCE TO PROMOTE NASAL BREATHING,” which is incorporated by reference herein in its entirety.

### FIELD OF THE INVENTION

The present invention generally relates to the field of medical devices. In particular, the present invention is directed to an apparatus and a method for sealing the oral cavity to promote nasal breathing.

### BACKGROUND

Nasal breathing devices are essential due to the significant health benefits associated with proper nasal respiration, including improved oxygen uptake, filtration of airborne particles, and humidification of inhaled air. Despite these benefits, many individuals suffer from nasal obstruction or conditions like sleep apnea, which hinder their ability to breathe effectively through the nose. Without adequate nasal breathing devices, these individuals often resort to mouth breathing, leading to issues such as dry mouth, increased risk of infections, and poor sleep quality. Current challenges include the discomfort, inefficiency, and lack of customization in existing nasal breathing solutions, highlighting the need for more effective and user-friendly devices.

### SUMMARY OF THE DISCLOSURE

In an aspect, an apparatus configured for sealing the oral cavity to promote nasal breathing. The apparatus includes a body having, a front surface, a rear surface, wherein the rear surface is positioned opposite the front surface, a perimeter, wherein the perimeter defines the boundary between the front surface and the rear surface, a first side, a second side, wherein the first side and the second side are located on opposite edges of the body, a lateral axis running from the first side to the second side, a first end, a second end, wherein the first end and the second end are located on opposite edges of the body, and a longitudinal axis running from the first end to the second end, wherein the body is configured to fit into a vestibular oral cavity of a subject, and a rim, wherein the rim is coupled to the perimeter of the body to provide stabilization and limit the body from migrating.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a diagram showing a front view of the apparatus according to one embodiment;

FIG. 2 is a diagram showing a rear view of the apparatus according to one embodiment;

FIG. 3 is a diagram showing an isometric view of the apparatus according to one embodiment;

FIGS. 4A, 4B, and 4C are diagrams showing alternative embodiments of a continuous standoff feature on the rear surface of the disclosed apparatus.

FIGS. 5A, 5B, and 5C are diagrams showing alternative embodiments of a segmented standoff feature on the rear surface of the disclosed apparatus.

FIG. 6A is a sectional isometric view showing a rim embodiment having a rounded profile extending from the front surface of the disclosed apparatus.

FIG. 6B is a sectional isometric view showing a rim embodiment having a bulbous profile extending outwardly from the front surface and the rear surface of the disclosed apparatus.

FIG. 6C is a sectional isometric view showing a rim embodiment having multiple spaced-apart flange skirts extending away from but inclined inwardly toward the front surface at the perimeter of the disclosed apparatus.

FIG. 6D is a sectional isometric view showing a rim embodiment having multiple spaced-apart flared projections extending away from but inclined outwardly away from the front surface at the perimeter of the disclosed apparatus.

FIG. 6E is a sectional isometric view showing a rim embodiment having a hooked shape that folds back toward the rear surface of the disclosed apparatus.

FIGS. 7A and 7B are diagrams showing frontal views of alternative embodiments of the disclosed apparatus having a single notch accommodating, respectively, superior and inferior labial frenula of the subject's mouth.

FIG. 7C is a diagram showing frontal views of alternative embodiments of the disclosed oval appliance having two notches that accommodate superior and inferior labial frenula of the subject's mouth and are of, respectively, greater and lesser concave extent.

FIG. 8A is a diagram showing a frontal view of the head of a subject having an exemplary apparatus emplaced in the vestibule of the subject's mouth.

FIG. 8B is a diagrammatic fragmentary side elevation view of the head of the subject of FIG. 8A, whose oral cavity is shown partly in cross section to indicate placement of the disclosed apparatus in the subject's mouth.

FIG. 9 is an exploded view of the disclosed apparatus in its modular state having separable components;

FIGS. 10A and 10B is a diagrammatic fragmentary side elevation view of the head of the subject of FIG. 8A, whose oral cavity is shown partly in cross section to indicate placement of the disclosed apparatus in the subject's mouth with the disclosed apparatus utilized in conjunction with an oral apparatus;

10C is an isometric illustration of the disclosed apparatus utilized in conjunction with an oral apparatus;
The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to an apparatus for sealing the oral cavity to promote nasal breathing. The apparatus includes a body having, a front surface, a rear surface, a perimeter, a first side, a second side, a lateral axis running from the first side to the second side, a first end, a second end, a longitudinal axis running from the first end to the second end, and a central region disposed between the first end and the second end, wherein the body is configured to fit into a vestibular oral cavity of a subject, and a rim, wherein the rim is coupled to the perimeter of the body to provide stabilization and limit the body from migrating.

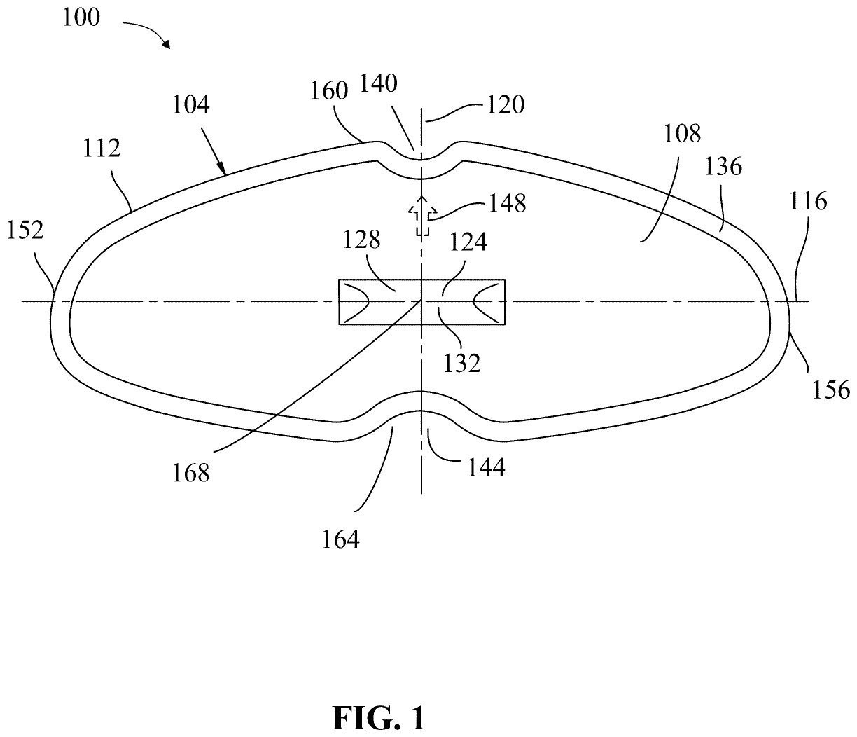

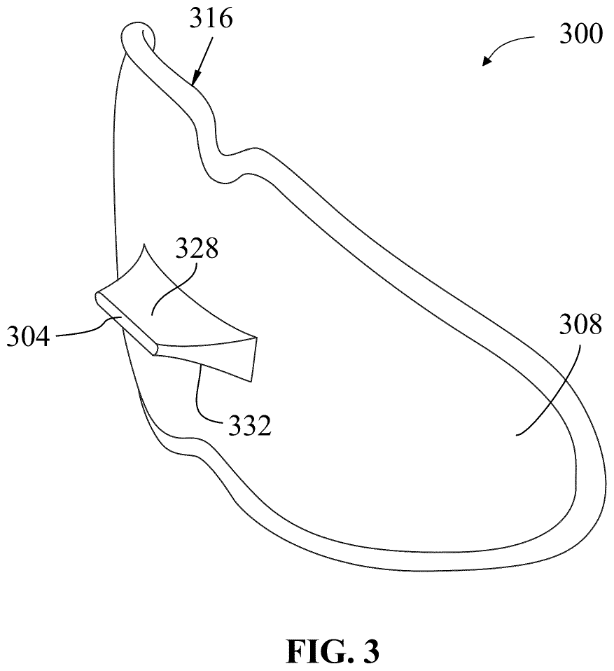

Referring now to FIG. 1, showing a frontal view of an embodiment of apparatus 100 wherein apparatus 100 includes body 104 having front surface 108, a rear surface, perimeter 112, first side 152, second side 156, lateral axis 116 running from first side 152 to second side 156, first end 160, second end 164, longitudinal axis 120 running from the first end 160 to second end 164, and central region 168 disposed between first end 160 and second end 164, wherein body 104 is configured to fit into a vestibular oral cavity of a subject. As used in this disclosure, a “body” is a main structure of an object. In a non-limiting example, apparatus 100 may be configured for placement into the vestibule of a subject's mouth and is sized accordingly to accommodate a range of anatomic variation across subjects. Without limitation, body 104 may vary in length along lateral axis 116 from about 90 mm to about 150 mm. Without limitation, body 104 may vary in length along longitudinal axis 120 from about 25 mm to about 75 mm. In a non-limiting example, the thickness of body 104 may vary from about 1 mm to about 3 mm. In a non-limiting example, body 104 may include a thin, flexible, elastomer material. Without limitation, body 104 may be made out of biocompatible silicone elastomer, thermoplastic polymers, such as nylon-based polymers (e.g., polyamide), thermoplastic elastomers (e.g., styrene-based copolymers), silicone-based materials, and the like. In a non-limiting example, body 104 may be flat. In another non-limiting example, the rear surface of body 104 may be concave in shape. Conversely, in another non-limiting example, the front surface 108 of body 104 may be concave in shape. As used in this disclosure, “concave” refers to the degree of inward curvature or depression on a specific surface. The concavity measurement of a surface may indicate how deeply the surface curves inward. A concavity measurement may include reference to the radius of curvature of a specific surface, wherein a smaller radius of curvature indicates a more pronounced concavity as the surface curves inward more sharply. Conversely, a concavity measurement may include a radius of curvature of a specific surface, wherein a larger radius of curvature may indicate a shallower concavity as the surface curves inward in a more gradual manner. In a non-limiting example, the concavity measurement may be used in the design and manufacture of body 104 of apparatus 100 to ensure apparatus 100 seamlessly fits on a user's dental arch. In a non-limiting example, body 104 concavity may measure 0 mm to 3 mm inward from the back surface as more clearly shown in the isometric view of apparatus 100 in FIG. 3. In a non-limiting example, body 104 may also have a concavity measurement from 0 mm to 3 mm inward from front surface 108. As used in this disclosure, a “perimeter” is the outer edge of an object. In a nonlimiting example, the perimeter of body 104 may include a flat smooth edge. As used in this disclosure, a “lateral axis” is the horizontal reference line that runs through an object from side to side. In a non-limiting example, lateral axis 116 may span from the left side of apparatus 100 to the right side of apparatus 100. In another non-limiting example, lateral axis 116 may be centrally located on body 104 and intersect with longitudinal axis 120. As used in this disclosure, a “longitudinal axis” is the reference vertical line that runs through an object from top to bottom. In a non-limiting example, longitudinal axis 120 may span the distance from the top of apparatus 100 to the bottom of apparatus 100. Without limitation, longitudinal axis 120 may be centrally located and dissect body 104 into two equal halves. In another non-limiting example, longitudinal axis 120 may intersect with lateral axis 116.

With continued reference to FIG. 1, apparatus 100 may further include tab 124, wherein tab 124 is coupled to front surface 108 of body 104 in the central region. As used in this disclosure, a “tab” is a feature of an object that projects from the surface used to grip the object. Without limitation, tab 124 is formed of opposite curved major surfaces 128 and 132. In an embodiment, tab 124 may be an optional feature in which apparatus 100 may or may not be included. In an embodiment, apparatus 100 is free of and does not include tab 124. Tab 124 is discussed in more detail in FIG. 3.

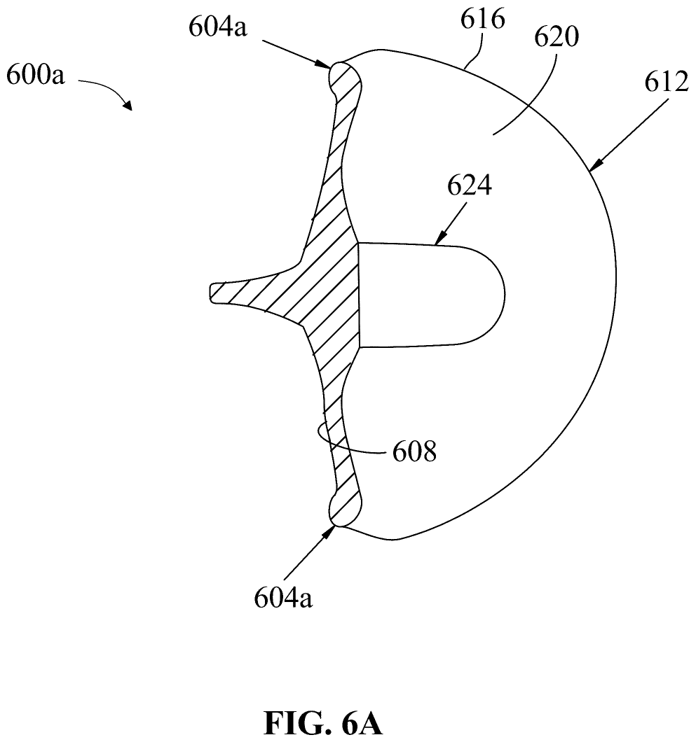

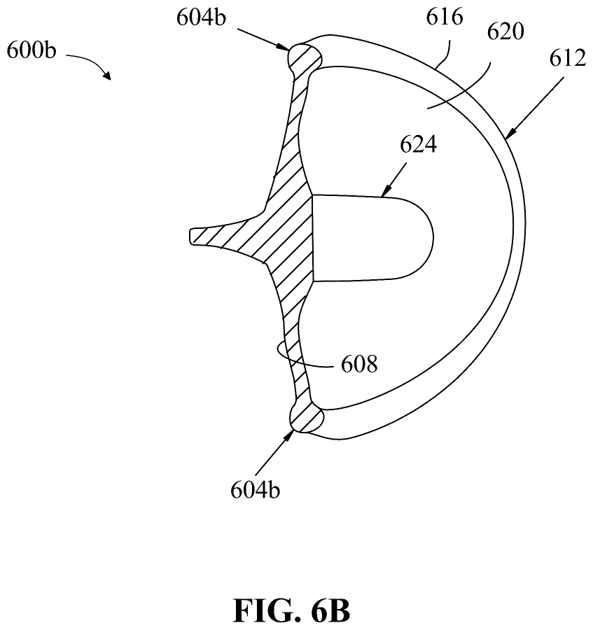

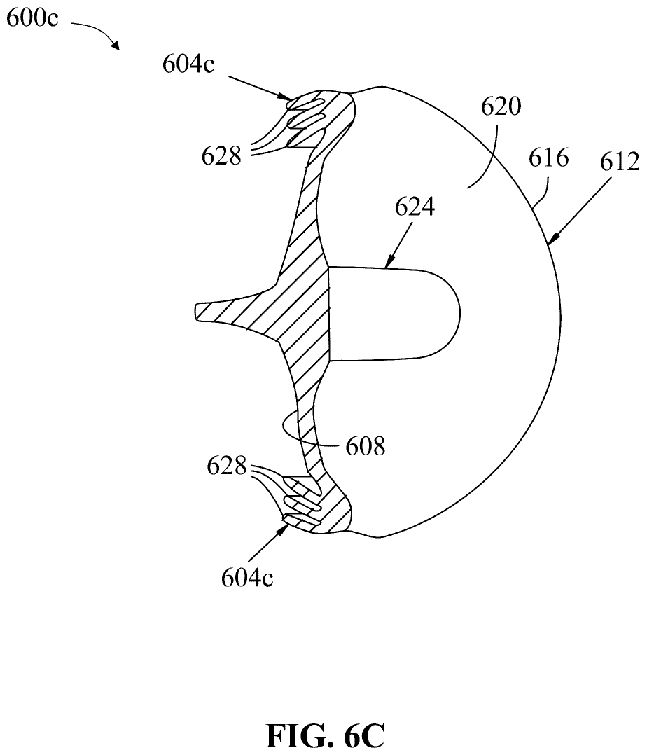

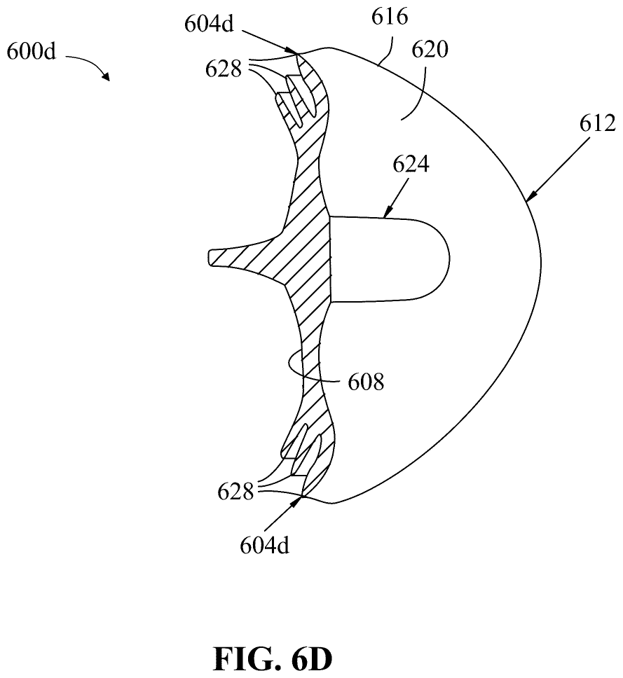

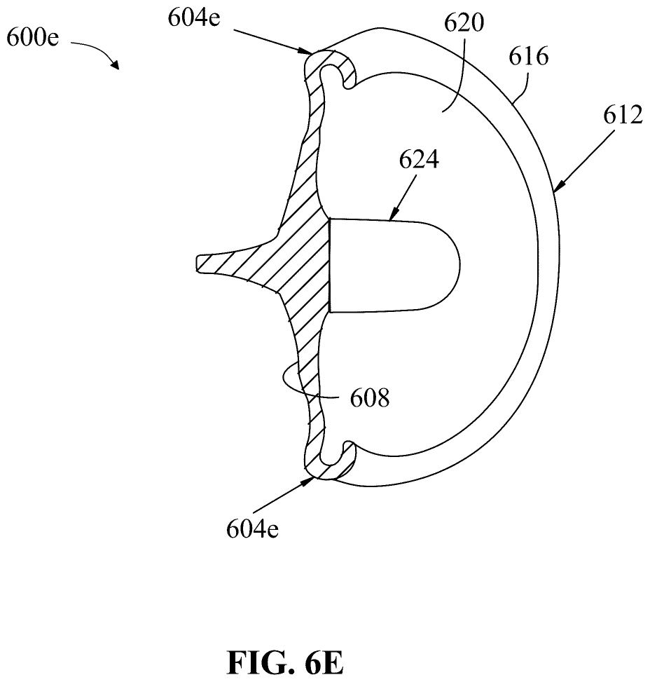

With continued reference to FIG. 1, without limitation, apparatus 100 further includes rim 136, wherein rim 136 is coupled to perimeter 112 of body 104 to provide stabilization and limit body 104 from migrating. As used in this disclosure, a “rim” is the outer most edge of an object that provides the object with a continuous form and boundary. In a non-limiting example, rim 136 may be raised between about 0 mm to about 1.5 mm. In a non-limiting example, rim 136 may include various shapes and sizes are defined in more detail in FIGS. 6A-6E. In another non-limiting example, rim 136 may be designed with a tapered shape, where the thickness of rim 136 progressively narrows from body 104 towards the edges. In a non-limiting example, a tapered shaped rim 136 may be characterized by a smooth, conical reduction in size, leading to a finer more pointed edge, to facilitate case of insertion and the like. In another nonlimiting example, rim 136 may be designed to have a bulbous shape, wherein rim 136 is rounded and protrudes outward in a convex manner. Continuing the previous, nonlimiting example, the bulbous shaped rim 136 may be characterized by a thickened, rounded chape, resembling a bulb or a swelling, thereby facilitating the grip, comfort, or the like of apparatus 100.

With continued reference to FIG. 1, without limitation, apparatus 100 further includes at least a notch feature as described in more detail below. As used in this disclosure, a “notch feature” is an area of an object where material is removed to create an indentation. In a non-limiting example, at least a notch feature may include superior notch 140 and inferior notch 144. As used in this disclosure, a “superior notch” is a notch that has material removed from the top surface of the object. As used in this disclosure, “inferior notch” is a notch that has material removed from the bottom surface of the object. In a nonlimiting example, superior notch 140 and inferior notch 144 may be included on apparatus 100 to avoid interference in the subject's superior labial frenulum and inferior labial frenulum. In an embodiment, apparatus 100 may include superior notch 140 and not inferior notch 144. In another embodiment, apparatus 100 may include inferior notch 144 and not superior notch 140. Without limitation, apparatus 100 may include both superior notch 140 and inferior notch 144. Without limitation, at least a notch may vary in width from about 5 mm to about 25 mm. Without limitation, at least a notch may vary in depth from about 5 mm to about 15 mm.

With continued reference to FIG. 1, without limitation, body 104 may include indicator 148. As used in this disclosure, an “indicator” is an element of an object that provides information regarding orientation of the object. In a non-limiting example, indicator 148 may include an arrow shape pointing in the direction of superior notch 140. Without limitation, indicator 148 may provide the subject with proper orientation of apparatus 100 utilizing tactile and visual means. Without limitation, indicator 148 may include an embossment, an engraving, and/or an area of increased surface roughness.

With continued reference to FIG. 1, without limitation, apparatus 100 may come in multiple sizes, for instance small and medium. Continuing the previous example, apparatus 100 size small may be recommended for female subjects and subjects with smaller jawlines to allow enough space for jaw movement without discomfort. Continuing the previous example, apparatus 100 size small may include an overall width of about 101.5 mm and a height of about 38 mm. Continuing the previous example, apparatus 100 size medium may be recommended for male subjects and subjects with broader jawlines to allow ample room for jaw motion. Continuing the previous example, apparatus 100 size medium may include an overall width of about 110 mm and a height of about 44.5 mm.

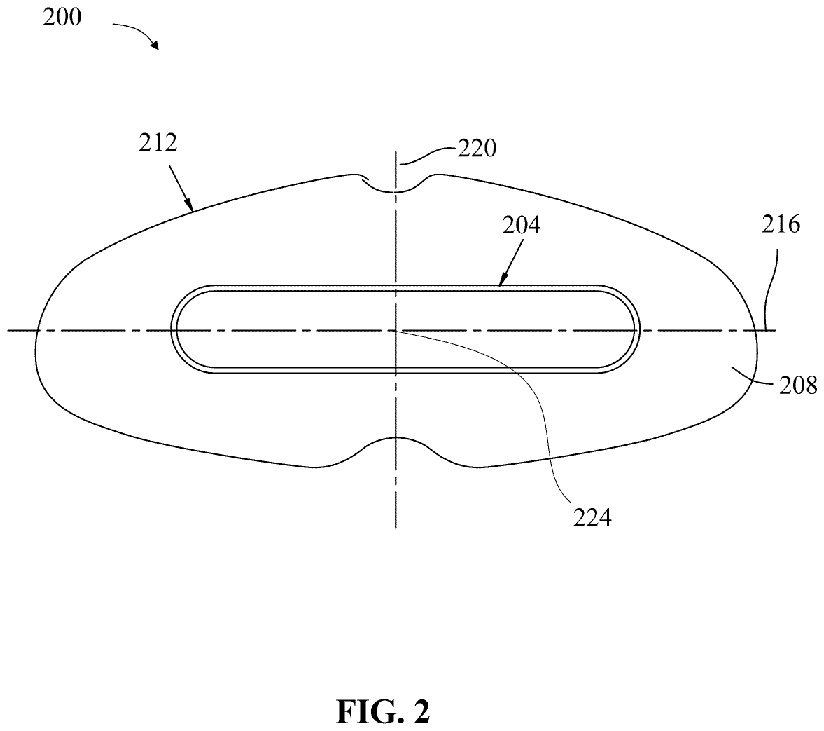

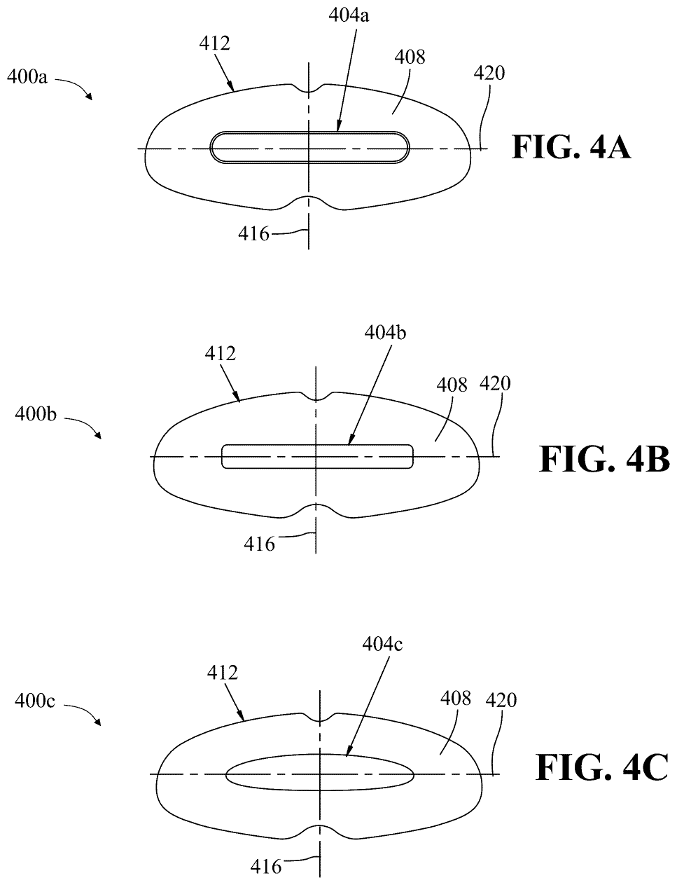

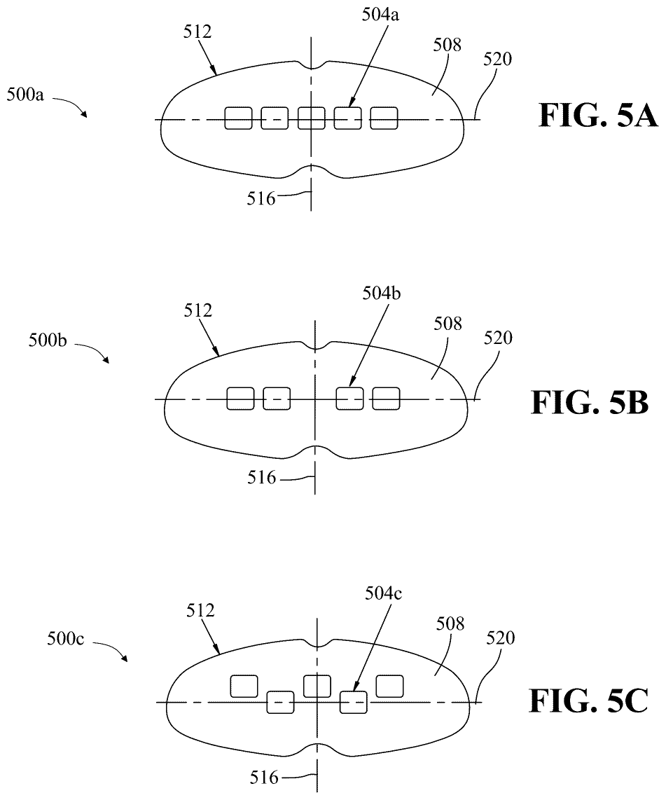

Referring now to FIG. 2, showing a rear view of an embodiment of apparatus 200 wherein apparatus 200 may include standoff feature 204, wherein standoff feature 204 is coupled to rear surface 208 of body 212 in central region 224 along lateral axis 216. As used in this disclosure, a “standoff feature” is an element of an object that protrudes away from the main body and is designed to provide a space between two surfaces and provide stability to apparatus 200. In a non-limiting example, standoff feature 204 may be designed to provide enhanced grip, or stability for apparatus 200 to not slip around on the subjects teeth and/or mouth. In a non-limiting example, standoff feature 204 may have a textured surface or a smooth surface. In a non-limiting example, standoff feature 204 may provide a distance between back surface 208 of body 212 and the teeth and/or gingivae of the subject wearing apparatus 200. In another non-limiting example, standoff feature 204 may include various configurations as further described in FIG. 4A-C and FIG. 5A-C. Without limitation, standoff feature 204 may be located in the central area of body 212 where lateral axis 216 and longitudinal axis 220 intersect. In another non-limiting example, standoff feature 204 may run the entire length of lateral axis 216 or just a partial length of lateral axis 216. Without limitation, standoff feature 204 may include one or more features of various shapes and sizes. In a non-limiting example, standoff feature 204 may include various lengths along lateral axis 216 from about 50 mm to 100 mm. In a non-limiting example, standoff feature 204 may include various lengths along longitudinal axis 220 from about 6 mm to 20 mm. In a non-limiting example, standoff feature 204 may include various projection distances away from rear surface 208 from about 0 mm to 10 mm.

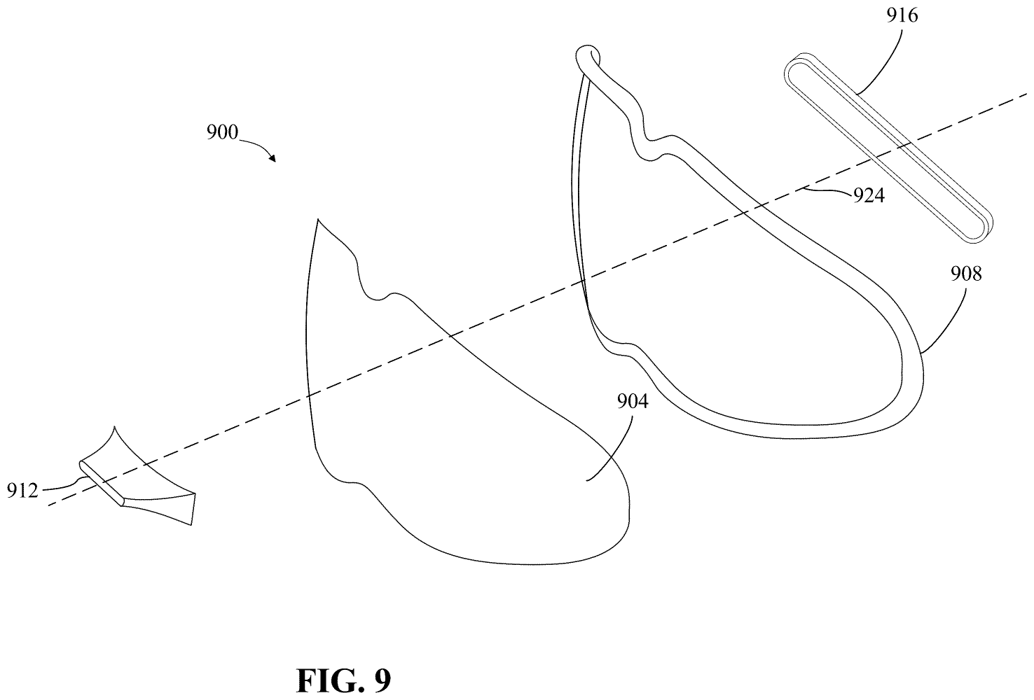

Referring now to FIG. 3, showing an isometric view of an embodiment of apparatus 300 wherein tab 304 may be configured to enable the subject to grip tab 304. In a non-limiting example, tab 304 projects from front surface 308 of body 316 a distance of about 5 mm to 7 mm. In a non-limiting example, tab 304 may be composed of the same or different material that is used for body 316. In a non-limiting example, tab 304 may be a modular component as discussed in FIG. 9.

Referring now to FIG. 4A-C are diagrams of apparatus 400a-c showing alternative embodiments of continuous standoff feature 404a-c on rear surface 408 of body 412. With reference to FIG. 4A-C, apparatus 400a-c includes standoff feature 404a-c which is made of a continuous pad material protruding from rear surface 408 of body 412.

With continued reference to FIG. 4, apparatus 400a depicts standoff feature 404a which is rectangular in shape with rounded corners. In an embodiment, standoff feature 404a is centrally located on rear surface 408 with the shape of standoff feature centered around the intersection of lateral axis 420 intersection with longitudinal axis 416. In an embodiment, standoff feature 404a may include longer opposite sides that run parallel to lateral axis 420 and shorter opposite sides that run parallel to longitudinal axis 416. In a non-limiting example, standoff feature 404a may include a hollowed out region in the center of the shape. In an embodiment, standoff feature 404a may protrude from rear surface 408 a distance of 0 mm to 10 mm. Without limitation, standoff feature 404a may provide a space or a gap between rear surface 408 of body 412 and the subjects teeth and/or gingivae when the subject is wearing apparatus 400a in the oral cavity.

With continued reference to FIG. 4, apparatus 400b depicts standoff feature 404b which is rectangular in shape with slightly rounded corners. In an embodiment, standoff feature 404b is centrally located on rear surface 408 with the shape of standoff feature centered around the intersection of lateral axis 420 intersection with longitudinal axis 416. In an embodiment, standoff feature 404b may include longer opposite sides that run parallel to lateral axis 420 and shorter opposite sides that run parallel to longitudinal axis 416. In a non-limiting example, standoff feature 404b may include a solid body, as opposed to a hollowed out shape (e.g., standoff feature of 404a). In an embodiment, standoff feature 404b may protrude from rear surface 408 a distance of 0 mm to 10 mm. Without limitation, standoff feature 404b may provide a space or a gap between rear surface 408 of body 412 and the subjects teeth and/or gingivac when the subject is wearing apparatus 400b in the oral cavity.

With continued reference to FIG. 4, apparatus 400c depicts standoff feature 404c which is oval in shape. In an embodiment, standoff feature 404c is centrally located on rear surface 408 with the shape of standoff feature centered around the intersection of lateral axis 420 intersection with longitudinal axis 416. In an embodiment, standoff feature 404c may include longer opposite sides that run parallel to lateral axis 420 and shorter, rounder sides of the oval shape running parallel to longitudinal axis 416. In a non-limiting example, standoff feature 404c may be a solid oval shape, as opposed to hollow (e.g., standoff feature 404a). In an embodiment, standoff feature 404c may protrude from rear surface 408 a distance of 0 mm to 10 mm. Without limitation, standoff feature 404c may provide a space or a gap between rear surface 408 of body 412 and the subjects teeth and/or gingivae when the subject is wearing apparatus 400c in the oral cavity.

Referring now to FIG. 5A-C, are diagrams of apparatus 500a-c showing alternative embodiments of noncontiguous standoff feature 504a-c on rear surface 508 of body 512. With reference to FIG. 5A-C, apparatus 500a-c includes standoff feature 504a-c which is made of noncontiguous pad material segments of substantially uniformed size, protruding from rear surface 508 of body 512.

With continued reference to FIG. 5, apparatus 500a depicts standoff feature 504a which is five noncontiguous pad material segments of substantially uniformed size and shape, protruding away from rear surface 508 in a straight alignment. As used in this disclosure, “substantially” means within a tolerance of +/−3 mm. In a non-limiting embodiment, standoff feature 504a is rectangular in shape with rounded corners. In another non-limiting example, standoff feature 504a may also be circular shaped, triangular shaped, star shaped, trapezoid shaped, hexagon shaped, pentagon shaped, and the like, with rounded edges to prevent damage to oral cavity. In an embodiment, standoff feature 504a is centrally located on rear surface 508 around the intersection of lateral axis 520 intersection with longitudinal axis 516 with a bias along lateral axis 520. In an embodiment, standoff feature 504a may include longer opposite sides that run parallel to lateral axis 520 and shorter opposite sides that run parallel to longitudinal axis 516. In an embodiment, standoff feature 504a may protrude from rear surface 508 a distance of 0 mm to 10 mm. Without limitation, standoff feature 504a may provide a space or a gap between rear surface 508 of body 512 and the subjects teeth and/or gingivae when the subject is wearing apparatus 500a in the oral cavity. In a non-limiting example, standoff feature 504a may be made of similar or the same material as body 512. In a non-limiting example, standoff feature 504a may be made of different material from body 512.

With continued reference to FIG. 5, apparatus 500b depicts standoff feature 504b which is four noncontiguous pad material segments of substantially uniformed size and shape, protruding away from rear surface 508 in a straight alignment. In a non-limiting embodiment, standoff feature 504b is rectangular in shape with rounded corners. In another non-limiting example, standoff feature 504b may also be circular shaped, triangular shaped, star shaped, trapezoid shaped, hexagon shaped, pentagon shaped, and the like, with rounded edges to prevent damage to oral cavity. In an embodiment, standoff feature 504b is centrally located on rear surface 508 around the intersection of lateral axis 520 intersection with longitudinal axis 516 with a bias along lateral axis 520. In an embodiment, standoff feature 504a may include longer opposite sides that run parallel to lateral axis 520 and shorter opposite sides that run parallel to longitudinal axis 516. In an embodiment, standoff feature 504b may protrude from rear surface 508 a distance of 0 mm to 10 mm. Without limitation, standoff feature 504b may provide a space or a gap between rear surface 508 of body 512 and the subjects teeth and/or gingivae when the subject is wearing apparatus 500b in the oral cavity. In a non-limiting example, standoff feature 504b may be made of similar or the same material as body 512. In a non-limiting example, standoff feature 504b may be made of different material from body 512.

With continued reference to FIG. 5, apparatus 500c depicts standoff feature 504c which is five noncontiguous pad material segments of substantially uniformed size and shape, protruding away from rear surface 508 in staggered alignment. In a non-limiting embodiment, standoff feature 504c is rectangular in shape with rounded corners. In another non-limiting example, standoff feature 504c may also be circular shaped, triangular shaped, star shaped, trapezoid shaped, hexagon shaped, pentagon shaped, and the like, with rounded edges to prevent damage to oral cavity. In an embodiment, standoff feature 504c is centrally located on rear surface 508 around the intersection of lateral axis 520 intersection with longitudinal axis 516 with a bias along lateral axis 520. In an embodiment, standoff feature 504c may include longer opposite sides that run parallel to lateral axis 520 and shorter opposite sides that run parallel to longitudinal axis 516. In an embodiment, standoff feature 504c may protrude from rear surface 508 a distance of 0 mm to 10 mm. Without limitation, standoff feature 504c may provide a space or a gap between rear surface 508 of body 512 and the subjects teeth and/or gingivae when the subject is wearing apparatus 500c in the oral cavity. In a non-limiting example, standoff feature 504c may be made of similar or the same material as body 512. In a non-limiting example, standoff feature 504c may be made of different material from body 512.

Referring now to FIG. 6A-E, apparatus 600 wherein rim 604a-e may include a plurality of configurations including, without limitation, a rounded profile, wherein the rounded profile extends from front surface 608 of body 612 perimeter 616, a bulbous profile, wherein the bulbous profile extends from both front surface 608 of body 612 perimeter 616 and rear surface 620 of body 612 perimeter 616, multiple flange skirts 628 extending toward front surface 608 of body 612 perimeter 616, multiple flared projections 628 extending away from front surface 608 of body 612 perimeter 616, and a hooked shape profile that folds back toward rear surface 620 of body 612.

With continued reference to FIG. 6A, is a cross-sectional view of apparatus 600a with rim 604a having the rounded profile extending from front surface 608. As used in this disclosure, a “rounded profile” is the smooth, continuous, curved shape of the outer contour of an object. In a non-limiting example, rim 604a may provide stabilization benefits for body 612 over alternative embodiments of other rims. In another non-limiting example, rim 604a may be made of the same or substantially the same material as body 612. In another non-limiting example, rim 604a may be composed of material that is different from body 612. In one or more embodiment, rim 604a may be a modular component and may be designed to be removed and swapped with another type of rim as disclosed herein. Without limitation, rim 604a may be smooth to the touch as to prevent irritation to oral cavity of the subject wearing apparatus 600a. Without limitation, rim 604a may rest up against the superior labial mucosa and the inferior labial mucosa.

With continued reference to FIG. 6B, is a cross-sectional view of apparatus 600b with rim 604b having the bulbous profile extending from both front surface 608 of body 612 and rear surface 620. As used in this disclosure, a “bulbous profile” is the smooth, continuous, curved shape of the outer contour of an object. In a non-limiting example, rim 604b may provide stabilization benefits for body 612 over alternative embodiments of other rims. In another non-limiting example, rim 604b may be made of the same or substantially the same material as body 612. In another non-limiting example, rim 604b may be composed of material that is different from body 612. In one or more embodiment, rim 604b may be a modular component and may be designed to be removed and swapped with another type of rim as disclosed herein. Without limitation, rim 604b may be smooth to the touch as to prevent irritation to oral cavity of the subject wearing apparatus 600b. Without limitation, rim 604b may rest up against the superior labial mucosa and the inferior labial mucosa.

With continued reference to FIG. 6C, is a cross-sectional view of apparatus 600c with rim 604c having multiple flange skirts 628 extending toward front surface 608. In a non-limiting example, rim 604c may provide stabilization benefits for body 612 over alternative embodiments of other rims. In another non-limiting example, rim 604c may be made of the same or substantially the same material as body 612. In another non-limiting example, rim 604c may be composed of material that is different from body 612. In one or more embodiment, rim 604c may be a modular component and may be designed to be removed and swapped with another type of rim as disclosed herein. Without limitation, rim 604c may be smooth to the touch as to prevent irritation to oral cavity of the subject wearing apparatus 600c. Without limitation, rim 604c may rest up against the superior labial mucosa and the inferior labial mucosa.

With continued reference to FIG. 6D, is a cross-sectional view of apparatus 600d with rim 604d having multiple flared projections 628 extending toward front surface 608. In a non-limiting example, rim 604d may provide stabilization benefits for body 612 over alternative embodiments of other rims. In another non-limiting example, rim 604d may be made of the same or substantially the same material as body 612. In another non-limiting example, rim 604d may be composed of material that is different from body 612. In one or more embodiment, rim 604d may be a modular component and may be designed to be removed and swapped with another type of rim as disclosed herein. Without limitation, rim 604d may be smooth to the touch as to prevent irritation to oral cavity of the subject wearing apparatus 600d. Without limitation, rim 604d may rest up against the superior labial mucosa and the inferior labial mucosa.

With continued reference to FIG. 6E, is a cross-sectional view of apparatus 600c with rim 604e a hooked shape that folds back toward rear surface 620 of body 612. In a non-limiting example, rim 604e may provide stabilization benefits for body 612 over alternative embodiments of other rims. In another non-limiting example, rim 604e may be made of the same or substantially the same material as body 612. In another non-limiting example, rim 604e may be composed of material that is different from body 612. In one or more embodiment, rim 604c may be a modular component and may be designed to be removed and swapped with another type of rim as disclosed herein. Without limitation, rim 604e may be smooth to the touch as to prevent irritation to oral cavity of the subject wearing apparatus 600c. Without limitation, rim 604c may rest up against the superior labial mucosa and the inferior labial mucosa.

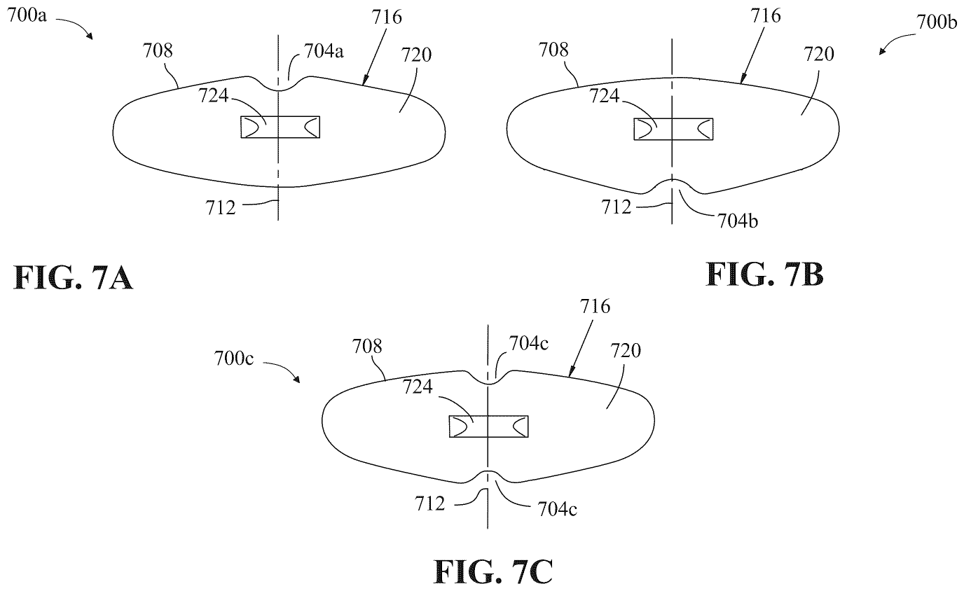

Referring now to FIG. 7A-C, apparatus 700a-c includes at least a notch feature 704 coupled to perimeter 708 and longitudinal axis 712 of body 716 wherein at least a notch feature 704 include a single notch to accommodate one labial frenulum, or two notches to accommodate both the superior labial frenulum and the inferior labial frenulum. With continued reference to FIG. 7A-C which shows front views of apparatus 700a-c with front surface 720 and tab 724. Without limitation, notch feature 704a-c may vary in width from about 5 mm to about 25 mm as previously mentioned above. Without limitation, at notch feature 704a-c may vary in depth from about 5 mm to about 15 mm, as previously mentioned above.

With continued reference to FIG. 7A which shows a frontal view of an embodiment of apparatus 700a having a single notch accommodating, the superior labial frenula of the subject's mouth. In a non-limiting embodiment, notch 704a may be centered around longitudinal axis 712. In another non-limiting embodiment, there may be material that separates notch 704a from tab 724. Without limitation, notch 704a may have rounded edges and notch 704a may be oval in shape. In a non-limiting example, notch 704a may be designed taking into consideration a subject's superior labial frenula dimensions for a custom fit or may be designed with a standard size.

With continued reference to FIG. 7B which shows a frontal view of an embodiment of apparatus 700b having a single notch accommodating the inferior labial frenula of the subject's mouth. In a non-limiting embodiment, notch 704b may be centered around longitudinal axis 712. In another non-limiting embodiment, there may be material that separates notch 704b from tab 724. Without limitation, notch 704b may have rounded edges and notch 704b may be oval in shape. In a non-limiting example, notch 704b may be designed taking into consideration a subject's superior labial frenula dimensions for a custom fit or may be designed with a standard size.

With continued reference to FIG. 7C which shows a frontal view of an embodiment of apparatus 700c having two notches that accommodate the superior labial frenula and the inferior labial frenula of the subject's mouth and are of, respectively, greater and lesser concave extent. In a non-limiting embodiment, notches 704c may be centered around longitudinal axis 712. In another non-limiting embodiment, there may be material that separates notches 704c from tab 724. Without limitation, notches 704c may have rounded edges and notches 704c may be oval in shape. In a non-limiting example, notches 704c may be designed taking into consideration a subject's superior labial frenula dimensions for a custom fit or may be designed with a standard size wherein the dimensions may be larger for the superior labial frenula and lesser for the inferior labial frenula.

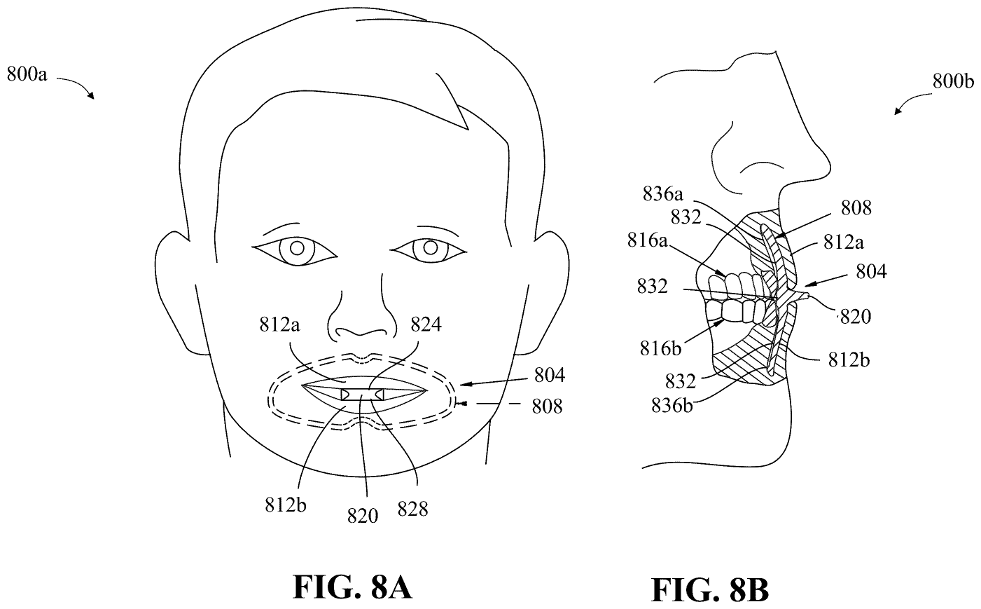

Referring now to FIG. 8A, is a diagram showing a frontal view of a subject's head 800a with apparatus 804 worn in the vestibule of the subject's mouth. Without limitation, body 808 of apparatus 804 is located behind upper lip 812a and lower lip 812b and upper teeth 816a and lower teeth 816b of the subject, with tab 820 protruding from the mouth such that the subject's lips 812a and 812b rest, respectively, on opposite curved major surfaces 824 and 828.

With continued reference to FIG. 8, FIG. 8B is a diagrammatic fragmentary side elevation view 800b of head 800a of the subject of FIG. 8A, whose oral cavity is shown partly in cross section to indicate placement of apparatus 804 in the subject's mouth. As shown, apparatus 804 is placed in the vestibule of the mouth, with body 808 positioned between lips 812a and 812b and teeth 816a and 816b. Without limitation, standoff feature 820 rests against teeth 816a and 816b to create a small gap between rear surface 832 of apparatus 804 and subject's teeth 816a and 816b and gums 836a and 836b. Without limitation, tab 820 protrudes from the mouth between subject's lips 812a and 812b. Without limitation, apparatus 804 may fit gently in front of the subject's teeth and allow the subject's jaw to relax slightly without air escaping the subject's mouth.

Referring now to FIG. 9 which shows an exploded view of apparatus 900 in its modular state having separable components. Without limitation apparatus 900 may be configured to be modular. As used in this disclosure, “modular” means that the object can be broken down into two or more components wherein the components can be replaced or interchanged. In a non-limiting example, body 904, rim 908, tab 912, and standoff 916 may be separable components as illustrated in FIG. 9, where the components are exploded along isometric axis 924.

With continued reference to FIG. 9, without limitation, apparatus 900 may include a plurality of components, wherein the component is designed to be easily interchanged and combined seamlessly with other components within the apparatus or other oral apparatus as discussed herein. In a non-limiting example, apparatus 900 components may include special grooves, channels, and other similar features that are designed for easily attaching and detaching the components. In a non-limiting example, apparatus 900 may include special grooves, channels, and other similar features that permit the subject to slide the components together for a seamless, precise fit of the overall assembly. In a non-limiting example, apparatus 900 may include features that attach to various moldable teeth parts. Without limitation, apparatus 900 components may piece together using a magnetic feature. In another non-limiting example, apparatus 900 components may be assembled and attached together using a molding technique. Continuing the previous example, without limitation, a molding technique may include heating each component separately, and pressing the components together in a mold to fuse the parts into a single, cohesive unit. Continuing the previous example, without limitation, the mold used to fuse the parts together may have the shape and size corresponding to the desired final output. Continuing the previous example, without limitation, each component may have thin film of material that, when heated, may adhere the components together. Continuing the previous example, without limitation, the final mold may be set to cool and the mold may retain structural integrity. In a non-limiting example, apparatus 900 may include features to permit the attachment of other oral apparatuses such as, without limitation, apparatus 900 may be attached to a mouth guard, or a dental tray, or braces, and the like. Without limitation, apparatus 900 may be able to attach to other oral apparatuses to facilitate the non-movement of apparatus 900 within the subjects mouth. For example, apparatus 900 may have a special groove designed on the body wherein the groove permits the user to attach their mouthguard to the back of apparatus 900 and lock apparatus 900 in place.

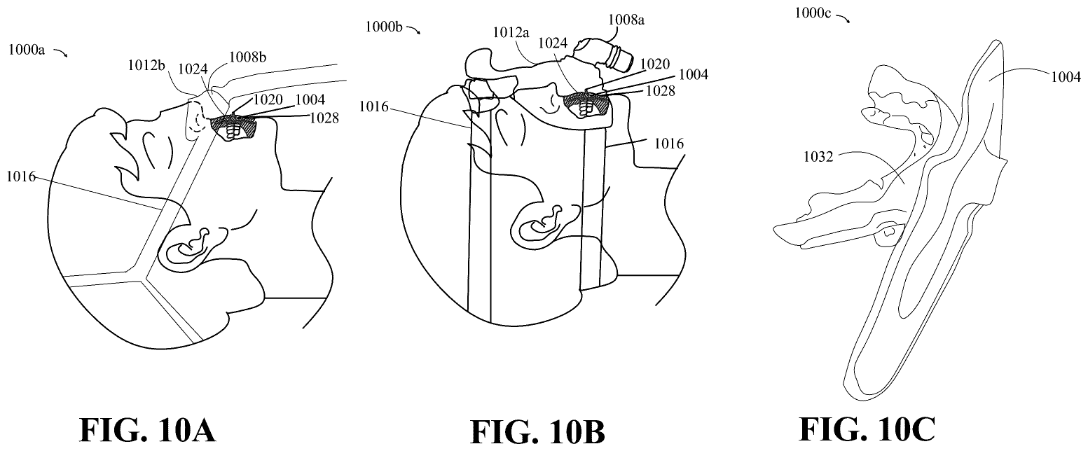

Referring now to FIGS. 10A and 10B, a diagrammatic fragmentary side elevation view of the head of the subject 1000a-b of FIG. 8A, whose oral cavity is shown partly in cross section to indicate placement of the disclosed apparatus in the subject's mouth with the disclosed apparatus 1004 utilized in conjunction with an oral apparatus 1008a-b. With continued reference to FIG. 10, FIG. 10C is an isometric illustration of the disclosed apparatus 1004 utilized in conjunction with an oral apparatus 1008c.

With continued reference to FIG. 10, as used in this disclosure, an “oral apparatus” is any device designed to interface with a subjects oral cavity, mouth, nasal passageway, nose, or face generally. In a non-limiting embodiment, oral apparatus 1008a-b may include a positive airway pressure therapy system, moldable teeth parts, any kind of oral appliances, mouth guards, nasal breathing strips, nasal prongs, nasal dilators, oxygen concentrators, ventilators, anti-snoring mouthpieces, tongue retaining devices, mandibular advancement devices (MADs), chin straps, breathing devices, orthodontic retainers, fluoride trays, braces, aligners, palatal expanders, and the like. In a non-limiting embodiment, oral apparatus 1008a-b may include a continuous positive airway pressure (CPAP) machine, a bilevel positive airway pressure (BiPAP) machine, an auto-adjusting positive airway pressure (APAP) machine, and the like. As used in this disclosure, a “CPAP machine” is a medical device used to treat obstructive sleep apnea and/or other sleep related breathing disorders by delivering a continuous flow of pressurized oxygen to the subject's airway. Without limitation, a CPAP machine may include mask 1012a-b, a motorized air compressor, tubing, filters, and control device. In a non-limiting example, mask 1012a-b used may be worn by the subject over the nose, over the mouth, over the mouth and nose, and the like, during sleep. As used in this disclosure, a “BiPAP machine” is a medical device used to treat obstructive sleep apnea and/or other sleep related breathing disorders by providing two levels of pressurized oxygen to the subject's airway. Similar to a CPAP machine, a BiPAP machine, without limitation, may include mask 1012a-b, a motorized air compressor, tubing, filters, and control device. Also similar to a CPAP machine, in a non-limiting example, mask 1012a-b used may be worn by the subject over the nose, over the mouth, over the mouth and nose, and the like, during sleep. Unlike the CPAP machine, a BiPAP machine provides two levels of pressure: a higher pressure when the subject inhales (e.g., inspiratory positive airway pressure (IPAP)) and a lower pressure when the subject exhales (e.g., expiratory positive airway pressure (EPAP)). Without limitation, a BiPAP machine may benefit a subject with different neuromuscular conditions and difficulties exhaling more than a CPAP machine which typically provides constant pressure throughout the breathing cycle. As used in this disclosure, an “auto-adjusting positive airway pressure (APAP) machine” is a medical device used to treat obstructive sleep apnea and/or other sleep related breathing disorders by providing dynamically adjusted pressurized oxygen to the subject's airway. Similar to a CPAP machine, an APAP machine, without limitation, may include mask 1012a-b, a motorized air compressor, tubing, filters, control device, one or more algorithms, pressure sensors, data storage capacity, a power supply, a display screen, and the like. In a non-limiting example, an APAP machine may also include connectivity to Wi-Fi and/or Bluetooth, and the like. Without limitation, an APAP machine may provide a more comfortable experience for the subject over a CPAP machine as the APAP machine continuously monitors the subject while sleeping and adjust pressure levels based on the subjects needs and sleep patterns and the CPAP machine, as described above, prescribes a constant pressure setting over the entire breathing cycle. In any case, with continued reference to FIG. 10, apparatus 1004 and oral apparatus 1008a-b may not create interferences. In a non-limiting embodiment, oral apparatus may include mask 1012a-b, wherein mask 1012a-b consists of rigid or semi rigid material which may be worn over the nose and/or mouth area. Mask 1012a-b may also include straps and/or fasteners 1016a-b to secure mask 1012a-b to the user's head in order to provide the pressurized air to the user's lungs. Conversely, apparatus 1004 rests inside the user's mouth with tab 1020 slightly revealed between the user's superior lip 1024 and inferior lip 1028. In a non-limiting embodiment, the revealed tab 1020 may protrude out from user's superior lip 1024 and inferior lip 1028 a rough distance 0 mm to 10 mm. In a non-limiting embodiment, when oral apparatus 1008a-b and apparatus 1004 are worn together, the oral apparatus 1008a-b sits on the exterior of the user's face, providing pressurized air to the user, and apparatus 1004 sits securely inside of the user's mouth to seal the oral cavity and promote nasal breathing. In a non-limiting example, apparatus 1004 encourages breathing through the nose thereby optimizing CPAP therapy efficiency.

With continued reference to FIG. 10, without limitation, apparatus 1004 is designed to enhance comfort and effectiveness during CPAP therapy by promoting nasal breathing and preventing mouth breathing. In a non-limiting example, apparatus 1004 may be made from a soft, flexible material that fits comfortably in a subject's teeth thereby blocking unwanted mouth airflow. In a non-limiting example, apparatus 1004 is designed to prevent the subject from having a dry mouth which may be associated with snoring and the use of a CPAP device.

With continued reference to FIG. 10, without limitation, as used in this disclosure, a “moldable teeth part” is a component that is designed to be worn in the oral cavity of a subject. In a non-limiting example, moldable teeth part 1032 may include thermoformable dental trays, retainers (e.g., metal retainers, plastic retainers, and the like), dental guards, dental prosthetics, braces, and the like. Without limitation, moldable teeth part 1032 may be worn in combination with apparatus 1004 and/or oral apparatus 1008a-b. In another non-limiting example, apparatus 1004 may be worn by a subject alone or in combination with moldable teeth parts and other mouth closure aids, such as, without limitation, chin straps and mouth tape, and the like. Without limitation, apparatus 1004 may not interfere with moldable teeth part 1032.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for sealing the oral cavity to promote nasal breathing, wherein the apparatus comprises:
a body having, a front surface, a rear surface, wherein the rear surface is positioned opposite the front surface, a perimeter, wherein the perimeter defines the boundary between the front surface and the rear surface, a first side, a second side, wherein the first side and the second side are located on opposite edges of the body, a lateral axis running from the first side to the second side, a first end, a second end, wherein the first end and the second end are located on opposite edges of the body, and a longitudinal axis running from the first end to the second end, wherein the body is configured to fit into a vestibular oral cavity of a subject; and
a rim, wherein the rim is coupled to the perimeter of the body to provide stabilization and limit the body from migrating; and
a standoff feature coupled to the rear surface of the body in the central region along the longitudinal axis and oval in shape.

2. The apparatus of claim 1, wherein the apparatus further comprises a central region disposed between the first end and the second end.

3. The apparatus of claim 1, wherein the apparatus further comprises a tab, wherein the tab is coupled to a front surface of the body in the central region.

4. The apparatus of claim 1, wherein the body further comprises an elastomer material.

5. (canceled)

6. The apparatus of claim 1, wherein the standoff feature comprises a continuous pad material protruding from the body.

7. The apparatus of claim 5, wherein the standoff feature comprises one or more noncontiguous, pad material segments of substantially uniform size.

8. The apparatus of claim 1, wherein the rim comprises a rounded profile, wherein the rounded profile extends from the front surface of the body.

9. The apparatus of claim 1, wherein the rim comprises a bulbous profile, wherein the bulbous profile extends from both the front surface of the body and the rear surface of the body.

10. The apparatus of claim 1, wherein the rim comprises multiple flange skirts extending toward the front surface of the perimeter.

11. The apparatus of claim 1, wherein the rim comprises multiple flared projections extending away from the front surface of the perimeter.

12. The apparatus of claim 1, wherein the rim comprises a hooked shape profile that folds back toward the rear surface of the body.

13. The apparatus of claim 1, wherein the rim comprises a tapered shape profile that gradually becomes narrow extending away from the perimeter.

14. The apparatus of claim 1, wherein the perimeter defines at least a notch feature.

15. The apparatus of claim 1, wherein the perimeter defines at least a notch feature, wherein the at least a notch feature comprises a single notch to accommodate the inferior labial frenulum.

16. The apparatus of claim 1, wherein the perimeter defines at least a notch feature, wherein the at least a notch feature comprises a single notch to accommodate the superior labial frenulum.

17. The apparatus of claim 1, wherein the perimeter defines at least a notch features, wherein the at least a notch feature comprises two notches to accommodate both the superior and inferior labial frenulum.

18. The apparatus of claim 1, wherein the apparatus comprises a plurality of components, wherein a component of the plurality of components is designed to be easily interchanged and combined seamlessly with other components of the apparatus utilizing at least a groove and channel.

19. The apparatus of claim 1, wherein the apparatus is configured to attach to an oral apparatus.

20. The apparatus of claim 19, wherein the oral apparatus comprises a positive airway pressure therapy system, moldable teeth parts, and nasal dilators.
